(19) United States
(12) Patent Application Publication
(10) Pub. No.: US 2026/0168990 A1
(43) Pub. Date: Jun. 18, 2026

(54) Evaluation Method for Potential Toxic Effect of Apolygus Lucorum Using In Vitro Tissue from Indoor Living Plant

(71) Applicant: Institute of Cotton Research of CAAS, Anyang (CN)

(72) Inventors: Junyu LUO, Anyang (CN); Jinjie CUI, Anyang (CN); Xiangzhen ZHU, Anyang (CN); Dongyang LI, Anyang (CN); Li WANG, Anyang (CN); Kaixin ZHANG, Anyang (CN); Jichao JI, Anyang (CN); Xueke GAO, Anyang (CN)

(21) Appl. No.: 18/983,123

(22) Filed: Dec. 16, 2024

(51) Int. Cl. G01N 33/50

(52) U.S. Cl. CPC G01N 33/5097

(57) ABSTRACT

An evaluation method for a potential toxic effect of Apolygus lucorum using an indoor living plant is provided, relating to the technical field of harmful organism toxicity determination and environmental safety evaluation of insect-resistant transgenic plants. The evaluation method includes determining a toxic effect of a chemical pesticide on the Apolygus lucorum and/or evaluating an effect of an insect-resistant transgenic plant on the Apolygus lucorum. The evaluation method can stably test the toxic effect of the chemical pesticide on the Apolygus lucorum by conducting multiple comparative analyses in indoor experiments, can comprehensively evaluate the effect of the insect-resistant transgenic plant on non-target organisms including the Apolygus lucorum, and can ensure that test insects receive sufficient nutrition, thereby extending an experimental period.

### TECHNICAL FIELD

The present disclosure relates to the technical field of harmful organism toxicity determination and environmental safety evaluation of insect-resistant transgenic plants, and in particular to an evaluation method for a potential toxic effect of Apolygus lucorum using an indoor living plant.

### BACKGROUND

The family Miridae is a series of phytophagous pests belonging to the order Hemiptera, with about five generations occurring a year. Their main dominant species include Apolygus lucorum, Adelphocoris fasciaticollis, and Adelphocoris suturalis, among which the Apolygus lucorum causes the most serious harm in most areas. The Apolygus lucorum is an omnivorous pest with main hosts being cotton, mulberry trees, jujube trees, grapes, hemp, beans, corn, potatoes, melons, alfalfa, medicinal plants, flowers, wormwood, and cruciferous vegetables.

Transgenic plants must undergo environmental safety assessment before environmental release or production testing. With the increasing number of novel transgenic insect-resistant materials, Apolygus lucorum has gradually become an important biological population for evaluating the environmental safety of transgenic crops. The dominant non-target insects of different plants are different, such that it is extremely important to select representative insects. The Apolygus lucorum is a dominant phytophagous organism in farmland as well as an important and representative phytophagous insect that meets the environmental safety assessment requirements of most transgenic crops. As transgenic insect-resistant plants are promoted, whether they may have an impact on phytophagous pests such as stink bugs, and whether this impact may be amplified along the food chain, thereby causing a greater impact on the entire farmland ecosystem, are key issues that must be evaluated before the promotion of transgenic crops.

The commonly used efficacy tests of chemical pesticides for phytophagous pests in farmland are the scintillation tube film method and the spot method. These two methods mainly adopt spraying or spotting to apply chemical pesticides to the body of phytophagous pests to produce a contact killing effect, but cannot achieve the evaluation for some non-contact killing chemical pesticides. In addition, these two methods do not provide direct nutritional supplements to phytophagous pests during the test, such that they can only be conducted in a highly short period of time and cannot realize long-term effects or chronic toxicity evaluations of some low-toxic and latently-toxic substances. Therefore, the scintillation tube film method and the spot method are not suitable for environmental safety assessment of transgenic plants.

Moreover, current research on the effects of transgenic insect-resistant plants on non-target organisms such as the Apolygus lucorum is mainly focused on field population dynamics surveys and molecular level studies of damage mechanisms, while resistance identification is also mainly conducted in the field. However, experiments conducted entirely in the field are greatly affected by natural conditions, and there are low repeatability and reliability of the identification results. Such experiments have a long cycle, increased time costs, and serious interference from other organisms. Especially in the case of complex interactions between food chains and ecosystems, it is difficult to obtain accurate evaluation results, and space and resources are limited. It is also difficult to fully cover the resistance performance in different environments and control variables. Furthermore, monitoring and recording are difficult and costly, while data analysis is difficult, such that it is difficult to obtain accurate results of environmental safety evaluation.

### SUMMARY

In view of this, the present disclosure provides an evaluation method for a potential toxic effect of Apolygus lucorum using an indoor living plant. The evaluation method can conduct environmental safety evaluation of indoor insect-resistant transgenic plants on non-target organisms including Apolygus lucorum under controllable laboratory conditions. The evaluation method has high stability, strong experimental controllability and applicability, and simple operation, and overcomes the shortcomings of conducting field experiments. Therefore, the evaluation method is a reliable method for evaluating the environmental safety of insect-resistant transgenic plants to non-target organisms including Apolygus lucorum.

The present disclosure provides an evaluation method for a potential toxic effect of Apolygus lucorum using an indoor living plant, including the following steps:
determining a toxic effect of a chemical pesticide on the Apolygus lucorum and/or evaluating a potential effect of an insect-resistant transgenic plant on a non-target organism including the Apolygus lucorum in a laboratory.

Preferably, the insect-resistant transgenic plant is at least one selected from the group consisting of insect-resistant transgenic soybean, insect-resistant transgenic wheat, and insect-resistant transgenic cotton.

The toxic effect of the chemical pesticide on the Apolygus lucorum is determined in the laboratory as follows:
spraying the chemical pesticide on an edible plant of the Apolygus lucorum to obtain a treatment group, taking the edible plant not sprayed with the chemical pesticide as a control group, collecting plant tissues from the treatment group and the control group and placing in an artificial insect-raising container, inoculating nymphs of the Apolygus lucorum as test insects on the plant tissues to allow a test, observing and recording growth and development of the test insects, and analyzing the toxic effect of the chemical pesticide on the Apolygus lucorum according to research purpose and requirement of the test; if the chemical pesticide does not produce an acute toxicity, observing a chronic toxicity of the chemical pesticide; if there is no significant difference in a development progress of the test insects in the treatment group and the control group, terminating the test 24 h after all the test insects in the control group have emerged; if there are a few test insects in the control group that have significantly slow growth and development, terminating the test when an emergence rate of the test insects in the control group reaches not less than 85% while remaining surviving test insects have not reached an age of 5.

The effect of the insect-resistant transgenic plant on the non-target organism including the Apolygus lucorum is evaluated in the laboratory as follows:
taking the insect-resistant transgenic plant not sprayed with the chemical pesticide as a treatment group, taking a non-transgenic plant not sprayed with the chemical pesticide as the control group, taking the non-transgenic plant sprayed with the chemical pesticide as a positive control group, collecting plant tissues from the treatment group, the control group, and the positive control group and placing in the artificial insect-raising container, inoculating the nymphs of the Apolygus lucorum as test insects on the plant tissues to allow a test, and observing and recording growth and development of the test insects; if there is no significant difference in a development progress of the test insects in the treatment group and the control group, terminating the test 24 h after all the test insects in the control group have emerged; if there are a few test insects in the control group that have significantly slow growth and development, terminating the test when an emergence rate of the test insects in the control group reaches not less than 85% while remaining surviving test insects have not reached an age of 5.

Preferably, the chemical pesticide in the positive control group is sprayed at a spraying amount when the chemical pesticide shows an optimal control effect on the Apolygus lucorum in the process for evaluating the effect of the insect-resistant transgenic plant on the Apolygus lucorum.

Preferably, the evaluation method is conducted in the artificial insect-raising container, and the artificial insect-raising container further includes a cotton ball fully immersed in honey water. Preferably, the honey water has a concentration of 10% to 15%.

Preferably, the nymphs are raised at 24° C. to 27° C. and a relative humidity of 60% to 80% under a photoperiod with 16 h of light and 8 h in the dark.

Compared with the existing technique, the evaluation method for toxicity can accurately control condition variables such as temperature, humidity, and light, avoiding interference caused by uncertainty in natural conditions in field trials and ensuring the repeatability and accuracy of the experiment. The experimental design enables the evaluation method to have a better toxicological evaluation effect on Apolygus lucorum, and is helpful to evaluate the potential risks of chemical pesticide or insect-resistant transgenic plant to organisms in the ecosystem. An experimental design of treatment group, negative control group, and positive control group is adopted. Through multiple comparisons, the effects of insect-resistant transgenic plant on non-target organisms such as Apolygus lucorum can be comprehensively evaluated. Meanwhile, the effectiveness and safety of pesticide control can also be compared, which is more scientific and accurate. Adding honey water to the insect-raising container ensures that the test insects can obtain sufficient nutrition, reduces the interference of hunger or malnutrition of the test insects on the experimental results, and prolongs the experimental period. This is particularly suitable for long-term observation of low toxicity or chronic toxicity of transgenic insect-resistant plant, breaking through the limitations of the evaluation method for contact chemical pesticide in the prior art. Dynamic observation of multiple parameters such as the growth and development, lifespan, and emergence of nymphs can comprehensively evaluate the effects of different treatments on the Apolygus lucorum, and reasonable experimental termination criteria are set based on the development of the test insects to ensure the integrity and accuracy of the experimental data. Compared with the existing method that relies on field trials, the evaluation method in laboratory simplifies the operating procedures, shortens the test cycle, reduces costs, and can obtain evaluation results in a relatively short time, thus providing a more efficient and scientific evaluation method for the environmental safety of transgenic insect-resistant plants. In summary, the evaluation method improves the accuracy and efficiency of evaluating the toxic effects of chemical pesticide on Apolygus lucorum and/or the effects of insect-resistant transgenic plant on Apolygus lucorum, while providing a more scientific experimental process. In this way, the evaluation method can better evaluate the toxic effects of chemical pesticide on Apolygus lucorum and/or the potential risks of transgenic crop to non-target organisms in the ecosystem.

### DETAILED DESCRIPTION OF THE EMBODIMENTS

To make the objective, technical solutions and advantages of the present disclosure clearer, the present disclosure will be further described below in detail below with reference to the examples. Unless otherwise defined, all technical terms used hereinafter have the same meaning as commonly understood by those skilled in the art. The technical terms used herein are merely for the purpose of describing specific examples, and are not intended to limit the protection scope of the present disclosure. Materials and instruments used in the following examples are all commercially available.

### Example 1

The insect-resistant transgenic plant materials and their corresponding non-transgenic plant materials were planted indoors or in the field under suitable temperature, humidity, light, and density, and it was ensured that the tested plants grew healthily and were not infected by any pests and diseases.

After the plants grew 2 to 4 leaves, the tested insect-resistant transgenic plants and their corresponding non-transgenic plant materials with basically the same growth trend were selected to collect their top tender leaves; when the tested plants were corn, corn kernels in the grain filling stage were collected for testing.

After the positive control group plants grew 2 to 4 leaves, the non-transgenic plants are sprayed with the recommended dose of Apolygus lucorum insecticide, and the spraying was uniform and complete on each plant, and the liquid did not drip on the leaf surface. After the liquid on the leaf surface was completely dried, leaves with basically the same growth were selected for testing. When the test plant was corn, the corn ear bracts were removed during the corn filling period, and the female ears were sprayed with the recommended dose of Apolygus lucorum insecticide uniformly and completely, and the liquid did not drip. After the liquid on the female ear was completely dried, the grains of basically the same size were selected for testing.

Cotton balls immersed in 15% honey water were added into a suitable container, and then placed in a suitable insect breeding container.

An appropriate amount of collected top tender leaves or corn kernels was put into artificial insect-raising containers, 1 Apolygus lucorum nymph of 1-day-age was inoculated into each container, and placed in an artificial climate box or artificial climate room at 24° C. to 27° C., a relative humidity of 60% to 80%, and a photoperiod of 16 h: 8 h (L: D). An appropriate amount of honey water was added every day, and fresh leaves or corn kernels from the same source were replaced according to the feeding consumption until the Apolygus lucorum nymphs in the control group emerged; each treatment was repeated at least 3 times, and the number of effective test insects in each repeat was not less than 30 nymphs.

The growth and development of the test insects were observed every day, and the number of surviving individuals, the development period of nymphs, lifespan, and the number of emerging adults was recorded.

If there was no significant difference in a development progress of the test insects in the treatment group of experimental system, the test was terminated 24 h after all the test insects in the control group had emerged; if there were a few test insects in the control group of experimental system that had significantly slow growth and development, the test was terminated when an emergence rate of the test insects in the control group reached not less than 85% while remaining surviving test insects had not reached an age of 5; and the sample was still included in the result calculation.

The results of growth and development parameters of Apolygus lucorum in different crop tissues were shown in Table 1.

TABLE 1
Results of growth and development parameters of Apolygus lucorum in different crop tissues.

| | | | | Male | Female |
|---|---|---|---|---|---|
| | | Nymph | | adult body | adult body |
| | Mortality | development | Emergence | weight | weight |
| Treatment | (%) | period d | rate % | mg | mg |
| 15% honey water | 100 | 7.09 | 0 | / | / |
| Transgenic corn kernels + | 27.88 | 10.02 | 90 | 6.25 | 6.28 |
| 15% honey water | | | | | |
| Transgenic soybean leaves + | 29.86 | 13.40 | 86.93 | 2.60 | 3.08 |
| 15% honey water | | | | | |
| Wheat leaves + | 25.86 | 13.02 | 84.26 | 2.18 | 2.75 |
| 15% honey water | | | | | |
| Transgenic cotton leaves + | 26.63 | 15.22 | 89.39 | 2.18 | 2.55 |
| 15% honey water | | | | | |
| Spraying cotton with emamectin | 100 | 0.3 | 0 | / | / |
| benzoate (30 mL/mu) + | | | | | |
| 15% honey water | | | | | |
| Spraying soybean with emamectin | 100 | 0.1 | 0 | / | / |
| benzoate (30 mL/mu) + | | | | | |
| 15% honey water | | | | | |

The technical features of the foregoing embodiments can be employed in arbitrary combinations. For brevity of description, not all possible combinations of the technical features of the foregoing embodiments are described. However, the combinations of the technical features should be construed as falling within the scope described in this specification as long as there is no contradiction in the combinations.

What is claimed is:

1. An evaluation method for a potential toxic effect of Apolygus lucorum using an indoor living plant, comprising the following steps:
determining a toxic effect of a chemical pesticide on the Apolygus lucorum and/or evaluating an effect of an insect-resistant transgenic plant on a non-target organism comprising the Apolygus lucorum in a laboratory; wherein
determining the toxic effect of the chemical pesticide on the Apolygus lucorum in the laboratory comprises:
spraying the chemical pesticide on an edible plant of the Apolygus lucorum to obtain a treatment group, taking the edible plant not sprayed with the chemical pesticide as a control group, collecting plant tissues from the treatment group and the control group and placing in an artificial insect-raising container, inoculating nymphs of the Apolygus lucorum as test insects on the plant tissues to allow a test, observing and recording growth and development of the test insects, and analyzing the toxic effect of the chemical pesticide on the Apolygus lucorum according to research purpose and requirement of the test; if the chemical pesticide does not produce an acute toxicity, observing a chronic toxicity of the chemical pesticide; if there is no significant difference in a development progress of the test insects in the treatment group and the control group, terminating the test 24 h after all the test insects in the control group have emerged; if there are a few test insects in the control group that have significantly slow growth and development, terminating the test when an emergence rate of the test insects in the control group reaches not less than 85% while remaining surviving test insects have not reached an age of 5; and
evaluating the effect of the insect-resistant transgenic plant on the non-target organism comprising the Apolygus lucorum in the laboratory comprises:
taking the insect-resistant transgenic plant not sprayed with the chemical pesticide as a treatment group, taking a non-transgenic plant not sprayed with the chemical pesticide as the control group, taking the non-transgenic plant sprayed with the chemical pesticide as a positive control group, collecting plant tissues from the treatment group, the control group, and the positive control group and placing in the artificial insect-raising container, inoculating the nymphs of the Apolygus lucorum as test insects on the plant tissues to allow a test, and observing and recording growth and development of the test insects; if there is no significant difference in a development progress of the test insects in the treatment group and the control group, terminating the test 24 h after all the test insects in the control group have emerged; if there are a few test insects in the control group that have significantly slow growth and development, terminating the test when an emergence rate of the test insects in the control group reaches not less than 85% while remaining surviving test insects have not reached an age of 5.

2. The evaluation method for a potential toxic effect of Apolygus lucorum using an indoor living plant according to claim 1, wherein the insect-resistant transgenic plant is at least one selected from the group consisting of insect-resistant transgenic soybean, insect-resistant transgenic wheat, and insect-resistant transgenic cotton.

3. The evaluation method according to claim 1, wherein the chemical pesticide in the positive control group is sprayed at a spraying amount when the chemical pesticide shows an optimal control effect on the Apolygus lucorum in the process for evaluating the effect of the insect-resistant transgenic plant on the non-target organism comprising the Apolygus lucorum.

4. The evaluation method according to claim 1, wherein the evaluation method is conducted in the artificial insect-raising container, and the artificial insect-raising container further comprises a cotton ball fully immersed in honey water.

5. The evaluation method according to claim 4, wherein the honey water has a concentration of 10% to 15%.

6. The evaluation method according to claim 1, wherein the nymphs are raised at 24° C. to 27° C. and a relative humidity of 60% to 80% under a photoperiod with 16 h of light and 8 h in the dark.
